(19) United States
(12) Patent Application Publication
(10) Pub. No.: US 2024/0417773 A1
(43) Pub. Date: Dec. 19, 2024

(54) METHOD FOR RAPIDLY DETERMINING SURVIVAL CONDITION OF SPORES

(71) Applicant: Zhejiang Cancer Hospital, Zhejiang (CN)

(72) Inventors: Wenjie WU, Zhejiang (CN); Ming ZHAO, Zhejiang (CN); Jinhui CHANG, Zhejiang (CN); Ming LIU, Zhejiang (CN); Yixuan JIN, Zhejiang (CN); Ali LI, Zhejiang (CN); Jie LYV, Zhejiang (CN)

(73) Assignee: Zhejiang Cancer Hospital, Zhejiang (CN)

(21) Appl. No.: 18/689,866

(22) PCT Filed: Apr. 11, 2023
(86) PCT No.: PCT/CN2023/087692

§ 371 Date: Mar. 7, 2024

(30) Foreign Application Priority Data
Apr. 19, 2022 (CN) 202210407807.1

(51) Int. Cl. C12Q 1/22; G01N 21/64

(52) U.S. Cl. CPC C12Q 1/22; G01N 21/6428

(57) ABSTRACT

The present disclosure discloses a method for rapidly determining survival condition of spores. The method includes the following steps: S100: performing sterilization treatment on the spore; S200: preparing a spore suspension with the spore subjected to the sterilization treatment; S300: dripping an appropriate amount of the above prepared spore suspension onto a solid medium, wherein 4-MUG is contained in the solid medium; S400: observing the solid medium under an excitation light source after the spore suspension fully infiltrates into the solid medium; and S500: determining the number of viable spores according to the number of the observed fluorescent spots on the solid medium. The method is based on an α-glucosidase test method and can rapidly determine activity of the spore and judge activity of a single spore.

### TECHNICAL FIELD

The present disclosure relates to the field of sterilization efficacy testing, in particular to a method for rapidly determining survival condition of spores.

### BACKGROUND

Some spore-forming bacteria may produce spores under unfavorable conditions (such as in lack of nutrients). Spores are super dormant structures that have high resistance to conventional sterilization means (high temperature, alcohol, strong oxidant, ultraviolet, chemical toxicant, etc). Therefore, spores are usually employed as a biological indicator for sterilization verification: a certain quantity of spores and a to-be-sterilized instrument are packaged separately and then put together in a sterilization device, after sterilization, whether the spores are killed is tested, if yes, it indicates that a sterilization process is up to standard, and otherwise, the sterilization process is below standard. At present, main photochemical methods commonly used for testing a survival condition of the spores are: a terbium-dipicolinic acid (Tb-DPA) test method, a microscopy-based endospore viability assay (micro-EVA) test method and an α-glucosidase test method.

The α-glucosidase test method is a method for indirectly determining activity of the spores by detecting activity of α-glucosidase, for the reason that the α-glucosidase is rich in spore coats of the spores and the activity of the α-glucosidase is well relevant to the activity of the spores. Specifically, 4-methylumbelliferyl-α-D-glucoside (4-MUG for short) is added in a to-be-tested sample liquid, though the 4-MUG has no fluorescence characteristic per se, 4-methylumbelliferone (4-MU for short), a product after being hydrolyzed by the α-glucosidase, has a fluorescence characteristic, and an emission spectrum of the 4-MU has a significant peak at 440 nm to 465 nm under an appropriate excitation light (an excitation wavelength is about 365 nm). Therefore, if the 4-MUG is added in the to-be-tested sample liquid, the activity of the α-glucosidase may be determined through a fluorescence intensity of the to-be-tested sample liquid, and then the activity of the spores is determined.

However, the current α-glucosidase test method is merely used for testing the spores in the sample liquid, so sensitivity is relatively low, an application scene is limited, and a single spore with activity cannot be tested.

### SUMMARY

### Technical Problem

In order to overcome the above defects in the prior art, the present disclosure provides a method for rapidly determining survival condition of spores. The method is based on an α-glucosidase test method and can rapidly determine the viability of the spore and determine viability of a single spore.

### Technical Solution

Technical problems to be solved by the present disclosure may be implemented through the following technical solutions.

A method for rapidly determining survival condition of spores includes the following steps:
S100: performing sterilization treatment on the spores;
S200: preparing a spore suspension with the spore subjected to the sterilization treatment;
S300: dripping an appropriate amount of the above prepared spore suspension onto a solid medium, wherein 4-MUG is contained in the solid medium.
S400: observing the solid medium under an excitation light source after the spore suspension fully infiltrates into the solid medium; and
S500: determining the number of viable spores according to the number of the observed fluorescent spots on the solid medium.

Further, the spore includes one or more types of spores among Bacillus atrophaeus (B. atrophaeus), Bacillus subtilis (B. subtilis), Geobacillus stearothermophilus (G. stearothermophilus), Bacillus pumilus (B. pumilus) and so one.

Further, a sterilization treatment method for the spore is a moist heat sterilization process, a dry heat sterilization process, a hydrogen peroxide sterilization process, an ethylene oxide sterilization process, a low-temperature steam formaldehyde sterilization process or other common sterilization processes.

Further, in step S100, the spore is carried on an object slide during sterilization.

Further, in step S200, the spore subjected to the sterilization treatment is resuspended in a buffer liquid or a recovered liquid and subjected to repeated washing, centrifugation, filtering and residue removal to obtain the spore suspension.

Further, components in the solid medium include: agar, tryptic soy broth and the 4-MUG.

Further, before step S300, the method further includes a step: preparing the solid medium.

Further, the step of preparing the solid medium is as follows:
mixing agar and tryptic soy broth with water in proportion and performing high-temperature sterilization to obtain a liquid medium;
dripping an appropriate amount of the liquid medium onto an object slide, and obtaining the solid medium after cooling and solidification; and
dropwise adding 4-MUG onto the solid medium in proportion, and waiting for the 4-MUG to infiltrate into the solid medium.

Further, in step S400, the step of observing the solid medium under the excitation light source is as follows:
S401: arranging a pad on a periphery of an observing region of the solid medium;
S402: covering the pad with a polydimethylsiloxane (PDMS) cover plate; and
S403: irradiating the observing region on the solid medium by using the excitation light source, and observing the solid medium in the observing region.

Further, a wavelength of the excitation light source is in a range from 340 nm to 380 nm.

### Beneficial Effects

The present disclosure has the following beneficial effects: the method for rapidly determining survival condition of spores based on the α-glucosidase test method, firstly, the 4-MUG is dropwise added onto the solid medium, then, the spore suspension is dripped onto the solid medium, the viable spore is induced to germinate and grow by using the still active α-glucosidase (if any) and nutrients in the solid medium, the viable spore is caused to synthesize sufficient α-glucosidase through metabolism, meanwhile, 4-MU, a product obtained after the 4-MUG is hydrolyzed by the α-glucosidase, is limited from being diffused outwards by means of the solid medium, so the hydrolysis product 4-MU is gathered on the periphery of each viable spore, each light spot corresponding to each viable spore is formed under the excitation light source, and not only may the activity of the spore be rapidly determined, but also the activity of the single spore may be determined.

### BRIEF DESCRIPTION OF FIGURES

FIG. 1 is a block diagram of steps of a method for quick determination the viability of the spore provided by the present disclosure.

FIG. 2 is a schematic diagram of a principle of a method for quick determination of the viability of the spore provided by the present disclosure.

### BEST MODE FOR CARRYING OUT THE INVENTION

### Detailed Description

The present disclosure is described in detail below with reference to the accompanying drawings and embodiments. Instances of the embodiments are shown in the accompanying drawings, and the same or similar reference numerals represent the same or similar elements or elements with the same or similar functions all the time. The embodiments described with reference to the accompanying drawings below are examples and are intended to explain the present disclosure but are not understood as a limitation to the present disclosure.

In the description of the present disclosure, it needs to be understood that direction or position relations indicated by terms such as “length”, “width”, “upper”, “lower”, “front”, “rear”, “left”, “right”, “vertical”, “horizontal”, “top”, “bottom”, “inner” and “outer” are direction or position relations as shown in the accompanying drawings and are only intended to conveniently describe the present disclosure and simplify the description but not to indicate or imply that a referred apparatus or element necessarily has a specific direction or is constructed or operated in the specific direction, so as not to be understood as a limitation to the present disclosure.

Besides, terms “first”, “second” and “third” are only used for description instead of being understood as indicating or implying a relative significance or implicitly indicating the quantity of referred technical features. Therefore, a feature limited by “first”, “second” and “third” may explicitly or implicitly include one or more of this feature. In the description of the present disclosure, “a plurality of” means two or more than two unless otherwise limited specifically clearly.

In the present disclosure, unless otherwise specified and limited clearly, terms such as “mount”, “connect”, “connection”, “secure” and “arrange” should be understood in a broad sense, for example, it may be a fixed connection or a detachable connection or integrated in whole; may be a mechanical connection or an electrical connection; or may be a direct connection, or an indirect connection through an intermediate medium, or communicating between insides of two elements or an interaction relation between the two elements. Specific meanings of the above terms in the present disclosure may be understood by those ordinarily skilled in the art according to specific conditions.

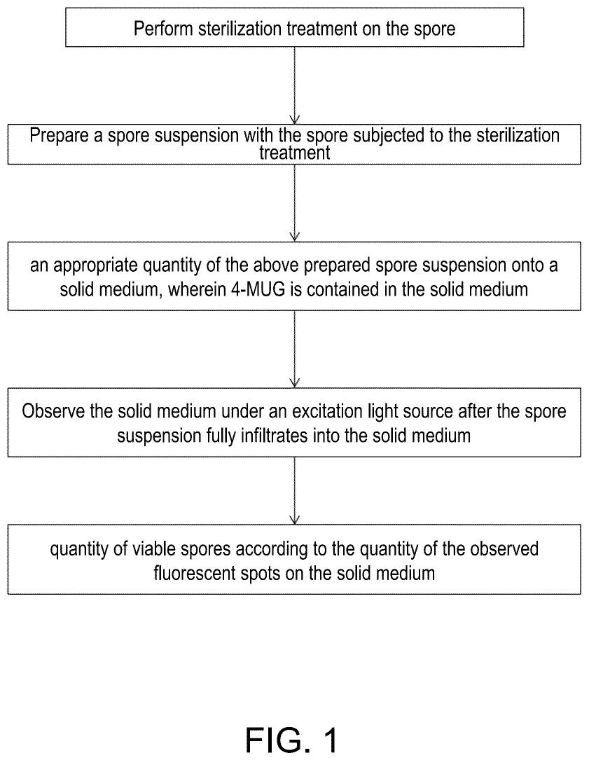

As shown in FIG. 1, a method for rapidly determining survival condition of spores includes the following steps:

S100: sterilization treatment is performed on the spores.

In step S100, the spore includes one or more types of spores among Bacillus atrophaeus (B. atrophaeus), Bacillus subtilis (B. subtilis), Geobacillus stearothermophilus (G. stearothermophilus), Bacillus pumilus (B. pumilus) and so on. A sterilization treatment method for the spore may be but not limited to a moist heat sterilization process, a dry heat sterilization process, a hydrogen peroxide sterilization process, an ethylene oxide sterilization process, a low-temperature steam formaldehyde sterilization process or other common sterilization processes.

A specific strain of the spore is determined depending on a specific sterilization treatment method, for example, if the sterilization treatment method adopts the ethylene oxide sterilization process, the spore includes one or more types of spores among Bacillus atrophaeus ATCC 9372, NCTC 10073, NCIMB 8058, DSM 2277, NRRL B-4418 or CIP 77.18; if the sterilization treatment method adopts the moist heat sterilization process, the spore includes one or more types of spores among Geobacillus stearothermophilus ATCC 7953 (NCTC 10007, DSM 22 and CIP 52.81) or ATCC 12980 (like NRRL B-4419); if the sterilization treatment method adopts the dry heat sterilization process, the spore includes one or more types of spores among Bacillus atrophaeus CIP 77.18, NCIMB 8058, DSM 675, NRRL B-4418 and ATCC 9372 or Bacillus subtilis DSM 13019; if the sterilization treatment method adopts the low-temperature steam formaldehyde sterilization process, the spore includes one or more types of spores among Geobacillus stearothermophilus NCIB 8224, DSM 6790, ATCC 10149 or ATCC 12980; and if the sterilization treatment method is the hydrogen peroxide sterilization process, the spore includes one or more types of spores among Geobacillus stearothermophilus ATCC 7953 or SSI K31.

It is regulated in “Sterilization of health care products—Biological indicators” that when the moist heat sterilization process or the low-temperature steam formaldehyde sterilization process is adopted, a viable bacteria count is not less than 1.0×10⁵, and when the ethylene oxide sterilization process, the hydrogen peroxide sterilization process or the dry heat sterilization process is adopted, the viable bacteria count is not less than 1.0×10⁶.

Preferably, the spore is carried on an object slide during sterilization.

S200: a spore suspension is prepared with the spore subjected to the sterilization treatment.

In step S200, the spore subjected to the sterilization treatment is put in a buffer liquid or a recovered liquid and subjected to repeated washing, centrifugation and residue removal to obtain the spore suspension.

The buffer liquid is a phosphate buffer liquid with a concentration of 0.03 M and pH of 7.2, and specific components and a preparation method of the phosphate buffer liquid belong to common general knowledge in the art and are thus not recorded in detail. The recovered liquid includes 0.1% polysorbate 80 and the above buffer liquid.

S300: an appropriate amount of the above prepared spore suspension is dripped onto a solid medium, wherein 4-MUG is contained in the solid medium.

In step S300, the amount of the spore suspension dripped onto the solid medium may be determined depending on specific test demands, and in this solution, only one or two drops of the spore suspension need to be dripped onto the solid medium.

The solid medium includes agar, tryptic soy broth (TBS) and the 4-MUG, wherein the agar is in a proportion of 30 g/L, the tryptic soy broth is in a proportion of 15 g/L, and a concentration of the 4-MUG in the solid medium is 50 mg/L to 500 mg/L.

Before step S300, the method further includes a step: preparing the solid medium.

The step of preparing the solid medium is as follows:
agar and tryptic soy broth are mixed with water in proportion and high-temperature sterilization is performed to obtain a liquid medium;
an appropriate amount of the liquid medium is dripped onto an object slide, and the solid medium is obtained after cooling and solidification; and
the 4-MUG is dropwise added onto the solid medium in proportion, and it waits for the 4-MUG to infiltrate into the solid medium.

The amount of the liquid medium dripped onto the object slide may be determined depending on specific test demands, and in this solution, only one or two drops of the liquid medium need to be dripped onto the object slide.

S400: the solid medium is observed under an excitation light source after the spore suspension fully infiltrates into the solid medium.

In step S400, the spore suspension on the solid medium is observed under an observation system. Viable spores in the spore suspension absorb all nutrients in the solid medium and then germinate and grow and synthesize sufficient α-glucosidase through metabolism, then the 4-MUG in the solid medium is hydrolyzed by the residual α-glucosidase (if any) after sterilization and α-glucosidase synthesized by the viable spores to generate a fluorescent indicator 4-MU, and the 4-MU generates fluorescent light under the excitation light source in a range from 340 nm to 380 nm.

Optimally, a wavelength of the excitation light source is about 365 nm.

In step S400, the step of observing the spore suspension on the solid medium under the excitation light source is as follows:

S401: a pad is arranged on a periphery of an observing region of the solid medium.

In step S401, the pad is supposed to surround the observing region, a region of the observing region may include an entire region on the solid medium where the spore suspension is dropwise added, or may also include a part of the region on the solid medium where the spore suspension is dropwise added, which is determined depending on specific observing demands.

In this solution, the pad is a rubber ring.

S403: the observing region on the solid medium is irradiated by using the excitation light source, and the solid medium in the observing region is observed.

S500: the number of viable spores is determined according to the number of the observed fluorescent spots on the solid medium.

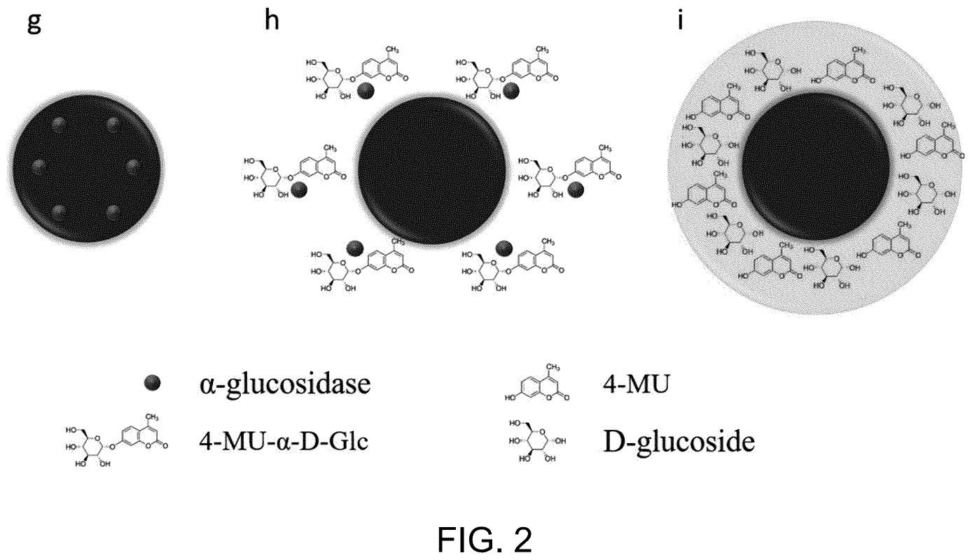

In step S500, as shown in FIG. 2, the 4-MUG is located in the solid medium and the 4-MU formed after the 4-MUG is hydrolyzed by the α-glucosidase is also limited by the solid medium and cannot be diffused outwards and then is gathered on the periphery of each viable spore, during observing under the excitation light source, a test person may observe light spots, one light spot represents a viable spore, the number of the viable spores on the solid medium is determined according to the number of the light spots, thus the total number of the viable spores during a whole sterilization treatment is deduced according to a proportion of dropwise adding the spore suspension, and finally, whether a sterilization process of the sterilization treatment is up to standard is evaluated.

The method for rapidly determining survival condition of spores based on the α-glucosidase test method, firstly, the 4-MUG is dropwise added onto the solid medium, then, the spore suspension is dripped onto the solid medium, the viable spores are induced to germinate and grow by using the still active α-glucosidase (if any) and nutrients in the solid medium, the viable spores are caused to synthesize sufficient α-glucosidase through metabolism, meanwhile, the 4-MU, a product obtained after the 4-MUG is hydrolyzed by the α-glucosidase, is limited from being diffused outwards by means of the solid medium, so the hydrolysis product 4-MU is gathered on the periphery of each viable spore, each light spot corresponding to each viable spore is formed under the excitation light source, and not only may the activity of the spores be rapidly determined, but also activity of a single spore may be determined.

This embodiment selects six 3M 1292 pressuresteam sterilization biological indicators (indicator). A bacterial carrier on each indicator includes about 106 spores of the Geobacillus stearothermophilus. The six indicators are divided into three groups, each of which contains two indicators. The biological indicators are put into a pressuresteam sterilization resistance detector (resistance detector) for the sterilization treatment, a sterilization temperature is 121° C., and a sterilization duration is 0 min, 8 min and 24 min respectively for a total of three groups. A treatment of 0 min at 121° C. means that no treatment is performed, and is a control group; a treatment of 8 min at 121° C. simulates a condition of incomplete sterilization; and treatment of 24 min at 121° C. simulates a condition of successful sterilization where all the spores are supposed to be skilled theoretically. Exposure positions of the indicators are located at a cold spot of a sterilization device. A sample subjected to the sterilization treatment is transferred onto a bechtop. Bacteria strips included in the three groups of biological indicators subjected to the sterilization treatment are taken out by means of sterilized tweezers, put in a sterile centrifuge tube, added with an appropriate amount of sterile recovered liquid (0.1% polysorbate 80, and 0.03 M phosphate buffer liquid with pH of 7.2), fully oscillated and crushed. Afterwards, the centrifuge tube is put in an ice-bath for cooling.

Then, a sample in the centrifuge tube is subjected to centrifugation (a centrifugal force of 2000×g, a temperature of 4° C. and a centrifugation duration of 20 min), an obtained supernatant passes through a 10 μm millipore filter (a spore diameter is 1 μm to 2 μm, so in order to make the spores pass through the filter and bacteria strip slags are blocked, a filter of 2 μm or above is needed), so as to obtain filtrate.

An appropriate amount of sterile recovered liquid is added into filtered slags (a main component is a crushed bacteria strip fiber) obtained after the above centrifugal filtration, after oscillating and shaking well, the above centrifugal filtration process is repeated, filtrate including thermal-damaged spores is further recovered, repetition is performed for 2 to 3 times like this, and the filtrate obtained by each centrifugal filtration is gathered as the spore suspension.

One drop of the liquid medium (30 g/L agar and 15 g/L tryptic soy broth) subjected to high-temperature sterilization is dropwise added onto an object slide, after this drop of the liquid medium is solidified, one drop of the 4-MUG with a concentration being 210 mg/L is dropwise added onto the solid medium, and a moment later, the 4-MUG fully infiltrates into the solid medium.

One drop of the spore suspension (including the thermal-damaged spores) is dropwise added onto the solid medium.

The number and sizes of fluorescent spots are observed under the observation system. The observation system is composed of four parts: 1) a stereoscopic microscope (a brand model may be: Nikon SMZ800); 2) a time gating camera (a brand model may be: Photonics Research Systems, Salford, UK) mounted on the above stereoscopic microscope; 3) a xenon flash lamp (a brand model may be: PerkinElmer, Waltham, MA) forming a 45° angle relative to a sample; and 4) a temperature-controlled microscope object slide stand (a brand model may be: Thermal).

Parameters set for the observation system are as follows: the xenon flash lamp has an excitation wavelength of 365 nm and an excitation slit width of 10 nm. Observed by the stereoscopic microscope, an emission wavelength is 544 nm, and an emission slit is 10 nm. The number and intensity of light spots are observed and results are shown in Table 1.

TABLE 1

| | Treatment | | |
|---|---|---|---|
| Number | condition | Observation result | Conclusion |
| A-1 | 121° C., | A large piece of overlapping | Unsterilized |
| | 0 min | fluorescent light and failure in | |
| | | recognizing existence of “spot” | |
| A-2 | 121° C., | A certain quantity of scattered | Incomplete |
| | 8 min | fluorescent spots | sterilization |
| A-3 | 121° C., | No fluorescent spot | Complete |
| | 24 min | | sterilization |

This embodiment selects six 3M 1295 hydrogen peroxide sterilization biological indicators (indicator). A bacterial carrier on each indicator includes about 106 spores of the Bacillus atrophaeus. The six indicators are divided into three groups, each of which has two indicators. The biological indicators are put into a hydrogen peroxide sterilization resistance detector (resistance detector) for the sterilization treatment, a sterilization temperature is 50° C., a hydrogen peroxide concentration is 1.74 mg/L, and a sterilization duration is 0 sec, 20 sec and 60 sec respectively for a total of three groups. A treatment for 0 sec at 50° C. means that no treatment is performed, and is a control group; a treatment for 20 sec at 50° C. simulates a condition of incomplete sterilization; and treatment for 60 sec at 50° C. simulates a condition of successful sterilization where all the spores are supposed to be skilled theoretically. An exposure position of the indicator is located at a cold spot of a sterilization device. A sample subjected to the sterilization treatment is transferred onto a bechtop. Bacteria strips included in the biological indicators in the three groups after the sterilization treatment are taken out by means of sterilized tweezers, put in a sterile centrifuge tube, added with an appropriate quantity of sterile recovered liquid (0.1% polysorbate 80, and 0.03 M phosphate buffer liquid with pH being 7.2), fully oscillated and crushed. Afterwards, the centrifuge tube is put in an ice-bath for cooling.

Then, a sample in the centrifuge tube is subjected to centrifugation (a centrifugal force being 2000×g, a temperature being 4° C. and a centrifugation duration being 20 min), an obtained supernatant passes through a 10 μm millipore filter (a spore diameter is 1 μm to 2 μm, so in order to make the spores pass through the filter and the bacteria strip slags are blocked, a filter of 2 μm or above is needed), so as to obtain filtrate.

An appropriate amount of sterile recovered liquid is added into filtered slags (a main component is a crushed bacteria strip fiber) obtained after the above centrifugal filtration, after oscillating and shaking well, the above centrifugal filtration process is repeated, filtrate including thermal-damaged spores is further recovered, repetition is performed for 2 to 3 times like this, and the filtrate obtained by each centrifugal filtration is gathered as the spore suspension.

One drop of the liquid medium (30 g/L agar and 15 g/L tryptic soy broth) subjected to high-temperature sterilization is dropwise added onto an object slide, after this drop of the liquid medium is solidified, one drop of the 4-MUG with a concentration being 210 mg/L is dropwise added onto the solid medium, and a moment later, the 4-MUG fully infiltrates into the solid medium.

One drop of the spore suspension (including the thermal-damaged spores) is dropwise added onto the solid medium.

The number and sizes of fluorescent spots are observed under the observation system. The observation system is composed of four parts: 1) a stereoscopic microscope (a brand model may be: Nikon SMZ800); 2) a time gating camera (a brand model may be: Photonics Research Systems, Salford, UK) mounted on the above stereoscopic microscope; 3) a xenon flash lamp (a brand model may be: PerkinElmer, Waltham, MA) forming a 45° angle relative to a sample; and 4) a temperature-controlled microscope object slide stand (a brand model may be: Thermal).

Parameters set for the observation system are as follows: the xenon flash lamp has an excitation wavelength of 365 nm and an excitation slit width of 10 nm. Observed by the stereoscopic microscope, an emission wavelength is 544 nm, and an emission slit is 10 nm. The number and intensity of light spots are observed and results are shown in Table 2.

TABLE 2

| | Treatment | | |
|---|---|---|---|
| Number | condition | Observation result | Conclusion |
| B-1 | 50° C., 1.74 | A large piece of overlapping | Unsterilized |
| | mg/L, 0 sec | fluorescent light and failure in | |
| | | recognizing existence of “spot” | |
| B-2 | 50° C., 1.74 | A certain quantity of scattered | Incomplete |
| | mg/L, 20 sec | fluorescent spots | sterilization |
| B-3 | 50° C., 1.74 | No fluorescent spot | Complete |
| | mg/L, 60 sec | | sterilization |

This embodiment selects six spore strips produced by Xinhua Mesa®Strip, which are divided into three groups and each group has two strips. The bacteria strips are put into a pressuresteam sterilization resistance detector (resistance detector) for the sterilization treatment, with a sterilization temperature of 121° C., and a sterilization duration of 0 min, 8 min and 24 min respectively for a total of three groups. A treatment for 0 min at 121° C. means that no treatment is performed, and is a control group; a treatment for 8 min at 121° C. simulates a condition of incomplete sterilization; and treatment for 24 min at 121° C. simulates a condition of successful sterilization where all the spores are supposed to be skilled theoretically. An exposure position of the indicator is located at a cold spot of a sterilization device. A sample subjected to the sterilization treatment is transferred onto a bechtop. Bacteria strips included in the biological indicators in the three groups after the sterilization treatment are taken out by means of sterilized tweezers, put in a sterile centrifuge tube, added with an appropriate amount of sterile recovered liquid (0.1% polysorbate 80, and 0.03 M phosphate buffer liquid with pH being 7.2), fully oscillated and crushed. Afterwards, the centrifuge tube is put in an ice-bath for cooling.

One drop of the liquid medium (30 g/L agar and 15 g/L tryptic soy broth) subjected to high-temperature sterilization is dropwise added onto an object slide, after this drop of the liquid medium is solidified, one drop of the 4-MUG with a concentration being 210 mg/L is dropwise added onto the solid medium, and a moment later, the 4-MUG fully infiltrates into the solid medium.

One drop of the spore suspension (including the thermal-damaged spores) is dropwise added onto the solid medium.

The number and sizes of fluorescent spots are observed under the observation system. The observation system is composed of four parts: 1) a stereoscopic microscope (a brand model may be: Nikon SMZ800); 2) a time gating camera (a brand model may be: Photonics Research Systems, Salford, UK) mounted on the above stereoscopic microscope; 3) a xenon flash lamp (a brand model may be: PerkinElmer, Waltham, MA) forming a 45° angle relative to a sample; and 4) a temperature-controlled microscope object slide stand (a brand model may be: Thermal).

Parameters set for the observation system are as follows: the xenon flash lamp has an excitation wavelength of 365 nm and an excitation slit width of 10 nm. Observed by the stereoscopic microscope, an emission wavelength is 544 nm, and an emission slit is 10 nm. The number and intensity of light spots are observed and results are shown in Table 3.

TABLE 3

| | Treatment | | |
|---|---|---|---|
| Number | condition | Observation result | Conclusion |
| C-1 | 121° C., | A large piece of overlapping | Unsterilized |
| | 0 min | fluorescent light and failure in | |
| | | recognizing existence of “spot” | |
| C-2 | 121° C., | A certain quantity of scattered | Incomplete |
| | 8 min | fluorescent spots | sterilization |
| C-3 | 121° C., | No fluorescent spot | Complete |
| | 24 min | | sterilization |

This embodiment selects six spore strips produced by Xinhua Mesa®Strip, which are divided into three groups and each group has two strips. The bacteria strips are put into a hydrogen peroxide sterilization resistance detector (resistance detector) for the sterilization treatment, a sterilization temperature is 50° C., a hydrogen peroxide concentration is 1.74 mg/L, and a sterilization duration is 0 sec, 20 sec and 60 sec respectively for a total of three groups. A treatment for 0 sec at 50° C. means that no treatment is performed, and is a control group; a treatment for 20 sec at 50° C. simulates a condition of incomplete sterilization; and treatment for 60 sec at 50° C. simulates a condition of successful sterilization where all the spores are supposed to be skilled theoretically. An exposure position of the indicator is located at a cold spot of a sterilization device. A sample subjected to the sterilization treatment is transferred onto a bechtop. Bacteria strips included in the biological indicators in the three groups after the sterilization treatment are taken out by means of sterilized tweezers, put in a sterile centrifuge tube, added with an appropriate amount of sterile recovered liquid (0.1% polysorbate 80, and 0.03 M phosphate buffer liquid with pH being 7.2), fully oscillated and crushed. Afterwards, the centrifuge tube is put in an ice-bath for cooling.

One drop of the liquid medium (30 g/L agar and 15 g/L tryptic soy broth) subjected to high-temperature sterilization is dropwise added onto an object slide, after this drop of the liquid medium is solidified, one drop of the 4-MUG with a concentration being 210 mg/L is dropwise added onto the solid medium, and a moment later, the 4-MUG fully infiltrates into the solid medium.

One drop of the spore suspension (including the thermal-damaged spores) is dropwise added onto the solid medium.

The number and sizes of fluorescent spots are observed under the observation system. The observation system is composed of four parts: 1) a stereoscopic microscope (a brand model may be: Nikon SMZ800); 2) a time gating camera (a brand model may be: Photonics Research Systems, Salford, UK) mounted on the above stereoscopic microscope; 3) a xenon flash lamp (a brand model may be: PerkinElmer, Waltham, MA) forming a 45° angle relative to a sample; and 4) a temperature-controlled microscope object slide stand (a brand model may be: Thermal).

Parameters set for the observation system are as follows: the xenon flash lamp has an excitation wavelength of 365 nm and an excitation slit width of 10 nm. Observed by the stereoscopic microscope, an emission wavelength is 544 nm, and an emission slit is 10 nm. The number and intensity of light spots are observed and results are shown in Table 4.

TABLE 4

| | Treatment | | |
|---|---|---|---|
| Number | condition | Observation result | Conclusion |
| D-1 | 50° C., 1.74 | A large piece of overlapping | Unsterilized |
| | mg/L, 0 sec | fluorescent light and failure in | |
| | | recognizing existence of “spot” | |
| D-2 | 50° C., 1.74 | A certain quantity of scattered | Incomplete |
| | mg/L, 20 sec | fluorescent spots | sterilization |
| D-3 | 50° C., 1.74 | No fluorescent spot | Complete |
| | mg/L, 60 sec | | sterilization |

It is to be noted finally that the above embodiments are merely for describing the technical solutions of the embodiments of the present disclosure but not for limiting them. Although the embodiments of the present disclosure have been described in detail with reference to the exemplary embodiments, those ordinarily skilled in the art are to understand that modifications or equivalent replacements may still be made for the technical solutions of the embodiments of the present disclosure. These modifications or equivalent replacements do not make modified technical solutions depart from the scope of the technical solutions of the embodiments of the present disclosure.

1. A method for rapidly determining survival condition of spores, comprising the following steps:
S100: performing sterilization treatment on the spore;
S200: preparing a spore suspension with the spore subjected to the sterilization treatment;
S300: dripping an appropriate amount of the above prepared spore suspension onto a solid medium, wherein 4-MUG is contained in the solid medium;
S400: observing the solid medium under an excitation light source after the spore suspension fully infiltrates into the solid medium; and
S500: determining the number of viable spores according to the number of the observed fluorescent spots on the solid medium.

2. The method for rapidly determining survival condition of spores according to claim 1, wherein the spore comprises one or more types of spores among Bacillus atrophaeus, Bacillus subtilis, Geobacillus stearothermophilus and Bacillus pumilus.

3. The method for rapidly determining survival condition of spores according to claim 1, wherein the sterilization treatment for the spore is a moist heat sterilization process, a dry heat sterilization process, a hydrogen peroxide sterilization process, an ethylene oxide sterilization process or a low-temperature steam formaldehyde sterilization process.

4. The method for rapidly determining survival condition of spores according to claim 1, wherein in step S100, the spore is carried on an object slide during sterilization.

5. The method for rapidly determining survival condition of spores according to claim 1, wherein in step S200, the spore subjected to the sterilization treatment is put in a buffer liquid or a recovered liquid and subjected to repeated washing, centrifugation, filtering and residue removal to obtain the spore suspension.

6. The method for rapidly determining survival condition of spores according to claim 1, wherein components in the solid medium comprise: agar, tryptic soy broth and the 4-MUG.

7. The method for rapidly determining survival condition of spores according to claim 1, wherein before step S300, the method further comprises a step: preparing the solid medium.

8. The method for rapidly determining survival condition of spores according to claim 7, wherein the step of preparing the solid medium is as follows:
mixing agar and tryptic soy broth with water in proportion and performing high-temperature sterilization to obtain a liquid medium;
dripping an appropriate amount of the liquid medium onto an object slide, and obtaining the solid medium after cooling and solidification; and
dropwise adding the 4-MUG onto the solid medium in proportion, and waiting for the 4-MUG to infiltrate into the solid medium.

9. The method for rapidly determining survival condition of spores according to claim 1, wherein in step S400, the step of observing the spore suspension on the solid medium under the excitation light source is as follows:
S401: arranging a pad on a periphery of an observing region of the solid medium;
S402: covering the pad with a polydimethylsiloxane cover plate; and
S403: irradiating the observing region on the solid medium by using the excitation light source, and observing the solid medium in the observing region.

10. The method for rapidly determining survival condition of spores according to claim 1, wherein a wavelength of the excitation light source is in a range from 340 nm to 380 nm.
